(19) United States
(12) Patent Application Publication
(10) Pub. No.: US 2026/0167682 A1
(43) Pub. Date: Jun. 18, 2026

(54) POLYPEPTIDE FOR PROMOTING ANGIOGENESIS AND COMPOSITION INCLUDING POLYPEPTIDE

(71) Applicant: Hybinis Biotechnology Limited, Hong Kong (CN)

(72) Inventors: Guiyi Gong, Hong Kong (CN); Mingyuen Lee, Hong Kong (CN); Qian Chen, Hong Kong (CN); Chen Zhao, Hong Kong (CN); Guangyi Fan, Hong Kong (CN)

(21) Appl. No.: 18/983,429

(22) Filed: Dec. 17, 2024

(51) Int. Cl. C07K 14/435; A61K 38/00; A61P 9/00; A61P 17/02; C12N 5/071

(52) U.S. Cl. CPC C07K 14/43595; A61P 9/00; A61P 17/02; C12N 5/069; A61K 38/00

(57) ABSTRACT

Provided are a polypeptide for promoting angiogenesis and a composition including the polypeptide. The polypeptide for promoting angiogenesis provided by the present application includes at least one selected from the group consisting of the following (a) to (c): (a) a polypeptide in which a carboxyl terminus has a GRPRF (SEQ ID NO: 12) sequence; (b) a polypeptide in which there is a GKR cleavage site at a carboxyl terminus; and (c) a polypeptide in which there is an amidation modification at a carboxyl terminus. By binding to NPY-Y2R of the neuropeptide Y (NPY) receptor family, the polypeptide ZsTx can regulate the proliferation, migration, and tube formation of vascular endothelial cells to promote the angiogenesis. The present application provides a brand-new target for the treatment of insufficient angiogenesis-associated diseases. The polypeptide ZsTx exhibits a strong pro-angiogenic activity, and has an activity concentration at a picomole level.

### TECHNICAL FIELD

The present application belongs to the technical field of biomedicine, and specifically relates to a polypeptide for promoting angiogenesis and a pharmaceutical composition including the polypeptide.

### REFERENCE TO SEQUENCE LISTING

The Sequence Listing XML file submitted via the USPTO Patent Center, with a file name of “Sequence listing_SCH-24104-USPT.xml”, a creation date of Dec. 9, 2024, and a size of 36,361 bytes, is part of the specification and is incorporated in its entirety by reference herein.

### BACKGROUND

Angiogenesis is a complicated multi-step process involving the activation, migration, proliferation, and differentiation of vascular endothelial cells, the degradation of basement membranes, and the formation of new vascular structures. Angiogenesis typically occurs during organism development, wound healing, and tissue regeneration. Angiogenesis is regulated by a balance of promoting and inhibiting factors to maintain the physiological homeostasis. Insufficient angiogenesis is an important cause of cardiovascular and peripheral artery diseases, including tissue ischemia, wound healing disorders, coronary artery disease, or the like. Currently, the common clinical pro-angiogenic therapies primarily involve the introduction of exogenous pro-angiogenic factors (such as vascular endothelial growth factor (VEGF) and basic fibroblast growth factor (bFGF)) into ischemic regions to increase the generation of collateral capillaries and establish a new capillary network. However, the traditional angiogenic factors as drugs have a short half-life, require repeated long-term administration, and may lead to adverse angiogenesis when used systemically, such as retinal neovascularization or the promotion of tumor growth. Therefore, there is an urgent clinical need to develop novel pro-angiogenic drugs and investigate new therapeutic targets.

Coral reef ecosystems, known for the high biodiversity and the productivity, are rich in natural resources and serve as marine drug banks for human health. As structural organisms in coral reef ecosystem, living corals are soft, benthic, and sessile. Due to the lack of physical defenses and delicate adaptive immune systems, corals have evolved a unique self-repair mechanism, which is conducive to the regeneration of the whole organism from tissue fragments and re-aggregated cells. Corals can repair their damaged parts through wound healing, tissue regeneration, and immune system reconstruction. It should be noted that angiogenesis is a key step involved in the wound healing of corals, suggesting the presence of key substances in corals that regulate angiogenesis. However, only a small number of pro-angiogenic active ingredients in enormous coral populations have been identified and thoroughly studied. For example, previous studies have shown that sesquiterpene compounds with new skeletons derived from soft corals have a pro-angiogenic activity, but their high effective concentration limits their potential for drug development.

### SUMMARY

The objective of the present application is to overcome the deficiencies of the prior art and provide a polypeptide for promoting angiogenesis and a composition containing the polypeptide.

In order to allow the above objective, the present application adopts the following technical solutions: a polypeptide for promoting angiogenesis, including at least one selected from the group consisting of the following (a) to (c):
(a) a polypeptide in which a carboxyl terminus has a GRPRF (SEQ ID NO: 12) sequence;
(b) a polypeptide in which there is a GKR cleavage site at a carboxyl terminus; and
(c) a polypeptide in which there is an amidation modification at a carboxyl terminus.

Preferably, a part of the polypeptide can form a α-helix secondary structure.

Preferably, the polypeptide is derived from a marine animal.

Preferably, the polypeptide has an amino acid sequence selected from the group consisting of the following: (1) ZsTx (SEQ ID NO: 1); (2) PcNPY (SEQ ID NO: 2); (3) LuNPY (SEQ ID NO: 3); and (4) PINPY (SEQ ID NO: 4).

One of the polypeptides for promoting angiogenesis is named ZsTx and has an amino acid sequence set forth in SEQ ID NO: 1.

The existing pro-angiogenic factors as drugs, such as VEGF and bFGF, have limitations including a short half-life, the need for repeated long-term administration, and potential adverse effects like promoting tumor growth when used systemically. Clinically, it is difficult to effectively control a dose and time, and the safety and efficacy need to be improved. The inventors of the present application have identified a novel soft coral-derived polypeptide ZsTx, which has an ability to specifically bind to NPY-Y2R of the neuropeptide Y (NPY) receptor family and an activity to significantly promote the angiogenesis at picomolar concentrations.

The present application also provides a nucleotide sequence encoding the polypeptide, where the nucleotide sequence is set forth in SEQ ID NO: 5.

The present application also provides a reagent for promoting angiogenesis, including the polypeptide ZsTx. Preferably, the reagent for promoting angiogenesis includes a reagent that promotes proliferation, tube formation, and/or migration of human umbilical vein endothelial cells. The inventors of the present application have found through experimental verification that the polypeptide ZsTx has the functions of promoting proliferation of human umbilical vein endothelial cells, promoting tube formation of human umbilical vein endothelial cells, and promoting migration of human umbilical vein endothelial cells, and thus can be used to prepare the reagent for promoting angiogenesis.

Preferably, a use concentration (or an activity concentration) of the polypeptide ZsTx is 1 pmol to 100 pmol.

The present application also provides a drug for preventing or treating a disease related to insufficient angiogenesis, including the polypeptide ZsTx.

In some such drugs for preventing or treating a disease related to insufficient angiogenesis, the disease related to insufficient angiogenesis includes a cardiovascular disease, a peripheral artery disease, a wound healing disorder, tissue ischemia, and a coronary artery disease.

Preferably, a use concentration (or an activity concentration) of the polypeptide ZsTx is 1 pmol to 100 pmol.

The present application also provides a pharmaceutical composition for promoting angiogenesis, including the polypeptide ZsTx and a pharmaceutically acceptable carrier.

In some such pharmaceutical compositions for promoting angiogenesis, a use concentration (or an activity concentration) of the polypeptide ZsTx is 1 pmol to 100 pmol.

The present application also provides a method for promoting angiogenesis, including: administering the polypeptide ZsTx to endothelial cells cultivated in vitro; or administering the polypeptide ZsTx to zebrafish or mice.

In the method for promoting angiogenesis, a concentration of the polypeptide ZsTx is 1 pmol to 100 pmol.

Preferably, the zebrafish undergoes an intersegmental vascular injury.

Preferably, the mice have a skin wound.

Compared with the prior art, the present application has the following beneficial effects: The present application provides a novel soft coral-derived polypeptide ZsTx. By binding to NPY-Y2R of the NPY receptor family, the polypeptide ZsTx of the present application can regulate the proliferation, migration, and tube formation of vascular endothelial cells to promote the angiogenesis. The present application provides a brand-new target for the treatment of insufficient angiogenesis-associated diseases. The polypeptide ZsTx of the present application exhibits a strong pro-angiogenic activity both in vitro and in vivo, and has an activity concentration at a picomole level, which is far superior to activity concentrations of the currently known coral-derived small-molecule compounds. Therefore, the polypeptide ZsTx of the present application exhibits high bioavailability and efficacy, and has a promising practical application prospect.

### BRIEF DESCRIPTION OF THE DRAWINGS

FIG. 1A to FIG. 1C show an amino acid sequence, a coding gene sequence, and evolutionary relationship analysis results for the polypeptide ZsTx, where FIG. 1A shows the full-length cDNA (SEQ ID NO: 5) and the amino acid sequence (SEQ ID NO: 1) for ZsTx, where the signal peptide sequence is MRAPTLAFFWLVGVVIFSTFSKSHS (SEQ ID NO: 9), the propeptide sequence is QQHRIEAIQLMASLDPEILATLSGSAEY (SEQ ID NO: 10), the NPY domain sequence is PDRPDVFKTPLQLRKYLESLNSFYALTGRPRF (SEQ ID NO: 11), and the precursor-associated amidation sequence is GKR;

FIG. 1B shows results of multiple sequence alignment of the polypeptide ZsTx with NPY/F polypeptides from different species; and

FIG. 1C shows a maximum likelihood tree for phylogenetic analysis of ZsTx with different NPY/F polypeptides from other species;

FIG. 2A to FIG. 2C show the verification of binding of ZsTx to NPY-Y2R through molecular docking and surface plasmon resonance (SPR) analysis, where

FIG. 2A shows a binding mode of ZsTx to NPY-Y2R, ZsTx (inside the dotted ellipse), and an extracellular structure of NPY-Y2R (outside the dotted ellipse), where the large dashed box is an enlarged view of the small dashed box; and in the large dashed box, binding sites of ZsTx to the extracellular structure of NPY-Y2R are shown, the secondary structure located above the two short dashed lines is a part of the extracellular structure of NPY-Y2R, and the secondary structure located below the two short dashed lines is a part of ZsTx;

FIG. 2B shows a secondary structure of ZsTx; and

FIG. 2C shows SPR analysis results of an interaction between ZsTx and NPY Y2R;

FIG. 3A to FIG. 3F show impacts of ZsTx on the proliferation, tube formation, and migration of human umbilical vein endothelial cells (HUVECs), where FIG. 3A shows a toxic effect of ZsTx for HUVECs;

FIG. 3B shows the proliferation of HUVECs that are starved for 24 h and then treated with ZsTx at different concentrations (1 pmol, 10 pmol, and 100 pmol) for 24 h, where *p<0.05 and **** p<0.0001 in contrast to a control group;

FIG. 3C shows results of a tube formation assay to observe an impact of ZsTx on the formation of a capillary network of HUVECs;

FIG. 3D shows quantitative analysis results of tube formation in different treatment groups, where data is expressed as mean±standard deviation (n≥3) of three independent experiments, and ** p<0.001 and *** p<0.001 in contrast to a control group;

FIG. 3E shows the promotion of ZsTx on migration of HUVECs in a wound healing experiment; and

FIG. 3F shows quantitative analysis results of wound closure rates in different treatment groups, where data is expressed as mean±standard deviation (n≥3) of three independent experiments, and *** p<0.001 in contrast to a control group;

FIG. 4A and FIG. 4B show a recovery effect of ZsTx for a vascular endothelial growth factor receptor tyrosine kinase inhibitor (VRI)-induced vascular injury in zebrafish, where FIG. 4A shows the growth of intersegmental blood vessels (ISVs) in a control group and zebrafish undergoing the VRI-induced vascular injury after treatments with ZsTx at different concentrations (1 pmol, 10 pmol, and 100 pmol);

FIG. 4B shows quantitative analysis results of the recovery of ISVs in zebrafish of each group, where data is expressed as mean±standard deviation (n=8), ####p<0.0001 in contrast to a control group, and **** p<0.0001 in contrast to a VRI treatment group;

FIG. 5A to FIG. 5D show the promotion of ZsTx on a wound healing process in mice.

FIG. 5A shows impacts of a control group and ZsTx treatment groups at different concentrations (1 ng/mL, 10 ng/mL, and 100 ng/mL) on a wound healing process in mice;

FIG. 5B shows wound healing ratios at different time points (day 0, day 2, day 4, day 6, and day 8), where data is expressed as mean±standard deviation (n=5), and ** p<0.01, *** p<0.001, and **** p<0.0001 in contrast to a control group;

FIG. 5C shows Masson staining results of collagen deposition in wound tissues of mice on day 8 after an injury (the scale bar=500 μm); and

FIG. 5D shows quantitative analysis results of collagen contents in mice of each group, where data is expressed as mean±standard deviation (n=5), and ** p<0.01 and **** p<0.0001 in contrast to the control group.

### DETAILED DESCRIPTION

The above contents of the present application are further described in detail below through specific implementations in the form of examples. However, it should not be understood that a scope of the above subject matter of the present application is limited merely to the following examples. In the examples, unless otherwise specified, the experimental methods used are conventional, and the materials and reagents used are commercially available.

### Example 1 Screening of ZsTx Polypeptide Sequences

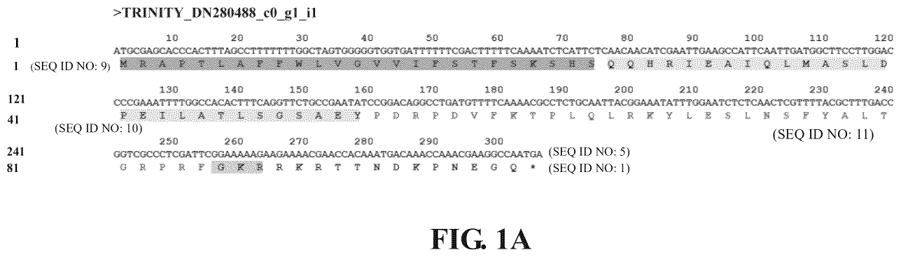

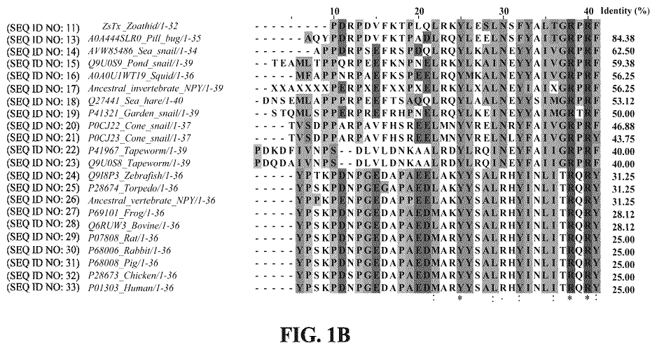

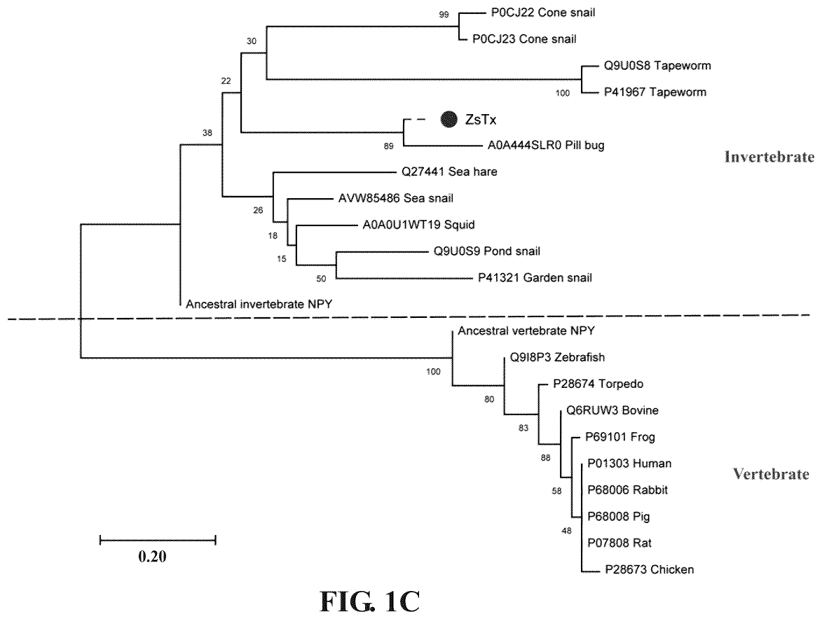

Through the Basic Local Alignment Search Tool (BLAST) software, a transcript fragment (ID: TRINITY_DN280408_c0_g1_i1.p1) was identified from a transcript sequence of the soft coral Zoanthus sociatus (Z. sociatus), and the transcript fragment was similar to a neuropeptide Y homolog (Uniprot ID: POCJ22) isolated from Conus betulinus. It was further verified by hmm-scan that there was a Hormone_3 domain (Pfam ID: PF00159) in the transcript fragment, which was a typical protein domain in different NPY/F polypeptides, with an expected accuracy of 0.98. In addition, repeated NCBI-BLASTP searches were conducted with an NPY/F-specific carboxyl-terminus sequence “-GRXRXGKR” (SEQ ID NO: 34, where X represents any amino acid, preferably a natural amino acid), and it was confirmed accordingly that the transcript fragment also had a similar sequence at a C-terminus. The transcript fragment was subjected to multiple sequence alignment and phylogenesis analysis with NPY/F homologs from different species, and as shown in FIG. 1A, FIG. 1B, and FIG. 1C, it was confirmed accordingly that ZsTx was a novel NPY analogue derived from a soft coral. It can be seen from the above that ZsTx is a novel invertebrate-derived NPY-homologous polypeptide. The complete primary structure sequence of ZsTx is as follows: MRAPTLAFFWLVGVVIFSTFSKSHSQQHRIEAIQLMASLDPEILATLSGSAEYPDRPDVFK TPLQLRKYLESLNSFYALTGRPRFGKRRKRTTNDKPNEGQ (SEQ ID NO: 1). The complete nucleotide sequence encoding ZsTx is as follows: 5′-ATGCGAGCACCCACTTTAGCCTTTTTTTGGCTAGTGGGGGTGGTGATTTTTTCGACT TTTTCAAAATCTCATTCTCAACAACATCGAATTGAAGCCATTCAATTGATGGCTTCCTT GGACCCCGAAATTTTGGCCACACTTTCAGGTTCTGCCGAATATCCGGACAGGCCTGA TGTTTTCAAAACGCCTCTGCAATTACGGAAATATTTGGAATCTCTCAACTCGTTTTAC GCTTTGACCGGTCGCCCTCGATTCGGAAAAAGAAGAAAACGAACCACAAATGACAA ACCAAACGAAGGCCAATGA-3′ (SEQ ID NO: 5).

The polypeptide of the NPY analogue derived from the soft coral is listed in Table 1.

TABLE 1

| | Amino acid | | Complete nucleotide sequence encoding the | |
|---|---|---|---|---|
| | sequence | | polypeptide | |
| Polypeptide | | SEQ ID | | SEQ ID |
| name | | NO: | | NO: |
| ZsTx | MRAPTLAFFW | 1 | 5′-ATGCGAGCACCCACTTTAGCCTTTTTTTGG | 5 |
| | LVGVVIFSTFS | | CTAGTGGGGGTGGTGATTTTTTCGACTTTTTC | |
| | KSHSQQHRIE | | AAAATCTCATTCTCAACAACATCGAATTGAAG | |
| | AIQLMASLDP | | CCATTCAATTGATGGCTTCCTTGGACCCCGAA | |
| | EILATLSGSAE | | ATTTTGGCCACACTTTCAGGTTCTGCCGAATA | |
| | YPDRPDVFKT | | TCCGGACAGGCCTGATGTTTTCAAAACGCCT | |
| | PLQLRKYLES | | CTGCAATTACGGAAATATTTGGAATCTCTCAA | |
| | LNSFYALTGRP | | CTCGTTTTACGCTTTGACCGGTCGCCCTCGAT | |
| | RFGKRRKRTT | | TCGGAAAAAGAAGAAAACGAACCACAAATG | |
| | NDKPNEGQ | | ACAAACCAAACGAAGGCCAATGA-3′ | |
| PcNPY | MKTLFVFLIVA | 2 | 5′-ATGAAACTGACCTGCGTGCTGATTATTGCG | 6 |
| | VFVANAAMYP | | GTGCTGTTTCTGACCGCGTGCCAGCTGACCA | |
| | PDRPDVFKTR | | CCGCGGAAACCTATAGCCGCGGCCGCCAGAA | |
| | ADLRQYLEEL | | ACATCGCGCGCGCCGCAGCACCGATAAAAAC | |
| | NEFYAITCGRP | | AGCAAATGGACCCGCACCTATCCGGATCCGC | |
| | RFGKRRKLSC | | CGGCGCGCCCGGCGGTGTTTCATAGCCGCGA | |
| | LTQCDERGCL | | AGAACTGATGAACTATCTGCGCGAACTGAAC | |
| | K | | CGCTATTTTGCGATTGTGGGCCGCCCGCGCTT | |
| | | | TGGCAAACGCGCGATTCTGGAAATTCATGTG | |
| | | | AAAAGCGGC-3′ | |
| LuNPY | MKLTCVLIIAV | 3 | 5′-ATGGCGGAAACCTATAGCCGCGGCCGCCAG | 7 |
| | LFLTACQLTTA | | AAACATCGCGCGCGCCGCAGCACCGATAAAA | |
| | ETYSRGRQKH | | ACAGCAAATGGACCCGCACCTATCCGGATCC | |
| | RARRSTDKNS | | GCCGGCGCGCCCGGCGGTGTTTCATAGCCGC | |
| | KWTRTYPDPP | | GAAGAACTGATGAACTATCTGCGCGAACTGA | |
| | ARPAVFHSREE | | ACCGCTATTTTGCGATTGTGGGCCGCCCGCGC | |
| | LMNYLRELNR | | TTTGGCAAACGCGCGATTCTGGAAATTCATGT | |
| | YFAIVGRPRFG | | GAAAAGCGGC-3′ | |
| | KRAILEIHVKS | | | |
| | G | | | |
| PlNPY | MASVRILALL | 4 | 5′-ATGGCGAGCGTGCGCATTCTGGCGCTGCTG | 8 |
| | MIGTVLMCQV | | ATGATTGGCACCGTGCTGATGTGCCAGGTGA | |
| | TANADSELLH | | CCGCGAACGCGGATAGCGAACTGCTGCATGA | |
| | EILSAHLEDN | | AATTCTGAGCGCGCATCTGGAAGATAACATG | |
| | MPVKRMLAPP | | CCGGTGAAACGCATGCTGGCGCCGCCGCAGC | |
| | QRPEEFKSPNE | | GCCCGGAAGAATTTAAAAGCCCGAACGAACT | |
| | LRKYLKAINE | | GCGCAAATATCTGAAAGCGATTAACGAATATT | |
| | YYAIVGRPRF | | ATGCGATTGTGGGCCGCCCGCGCTTTGGCAA | |
| | GKRPALRLQA | | ACGCCCGGCGCTGCGCCTGCAGGCGCGCCGC- | |
| | RR | | 3′ | |

### Example 2 Determination of a Target of the Polypeptide ZsTx

### 1. Molecular Docking

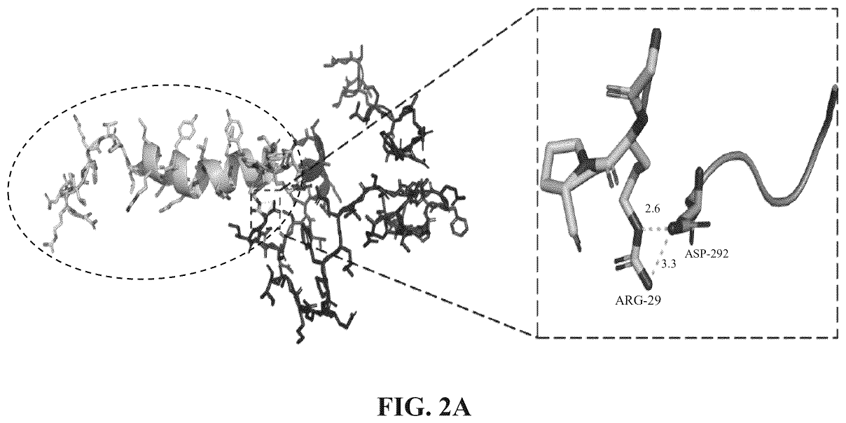

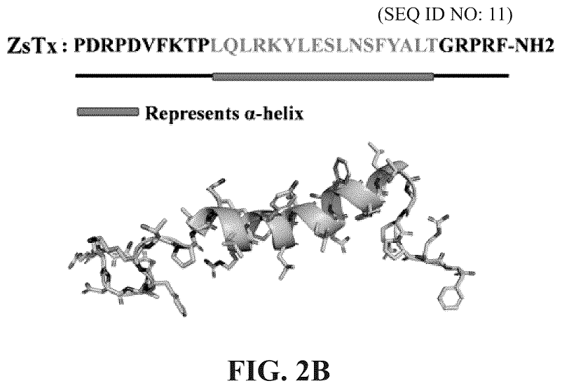

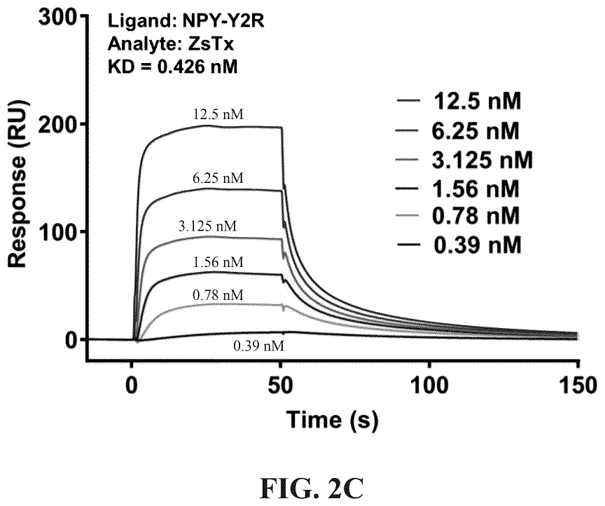

A structure of an NPY Y2R receptor domain was acquired from an RCSB protein data bank (PDB code: 7ddz). The structure of ZsTx was constructed by the Swiss model website. The structure was verified with the PyMOL 2.4.1 software. A structure diagram was finally plotted. Molecular docking was conducted with the Zdock software. The cluster analysis was conducted for docking results according to a root mean square deviation (RMSD) of the polypeptide. A conformation with the lowest energy in the largest cluster was considered the optimal docking conformation for analysis. Asshown in FIG. 2A and FIG. 2B, ZsTx has a potential to bind to NPY-Y2R.

### 2. SPR

The specific experimental method was as follows: 0.4 M 1-ethyl-3-(3-dimethylaminopropyl) carbodiimide hydrochloride and 10 mM N-hydroxysuccinimide were used to activate a CM5 sensor chip for 20 min at a flow rate of 5 μL/min. An NPY-Y2R protein was diluted with 1 mL of sodium acetate (10 mM, pH 5.0) to 20 μg/mL and then allowed to flow over an activated surface, so as to allow a target response value (RU). The remaining activation sites on the sensor chip were blocked with 45 μL of hexanolamine (1 M, pH 8.5). Real-time test results were recorded with a Biacore S200 instrument (United States) at a flow rate of 30 μL/min. Then, in order to determine an equilibrium dissociation constant (KD) of NPY Y2R and ZsTx, ZsTx serially diluted (10 μM, 5 μM, 2.5 μM, and 1.25 μM) was treated in a 150 mM phosphate buffered saline (PBS) at pH 7.4, and an interaction between ZsTx and NPY Y2R immobilized on the surface of the sensor chip was analyzed with the BIA evaluation program. As shown in FIG. 2C, ZsTx can bind to NPY-Y2R with a KD constant of 0.426 nM.

### Example 3 Verification of a Pro-Angiogenic Activity of the Polypeptide ZsTx for HUVECs In Vivo

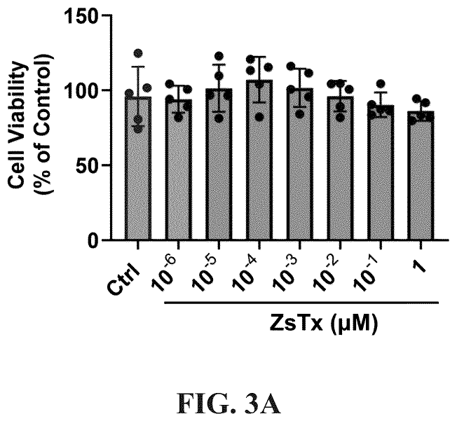

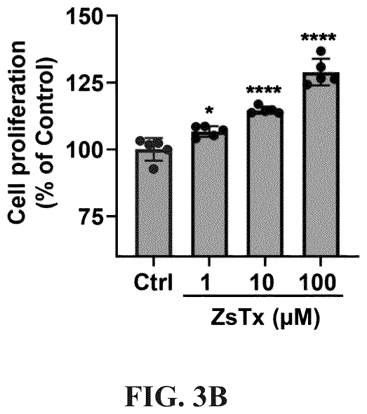

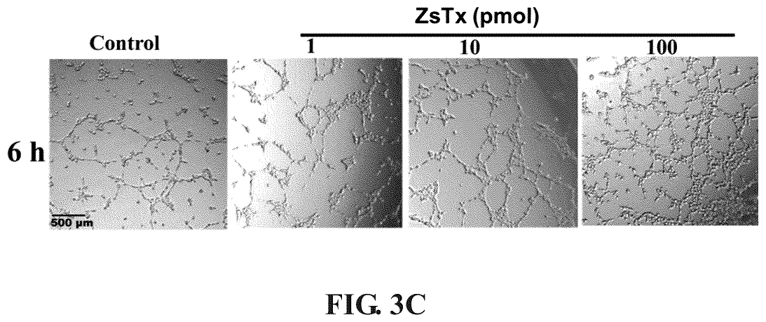

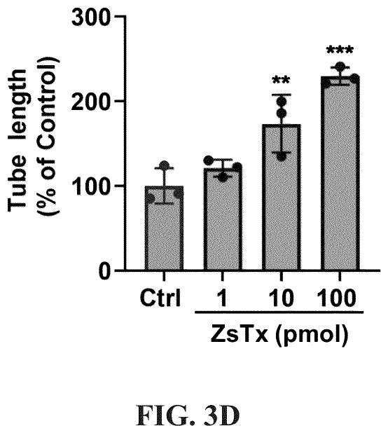

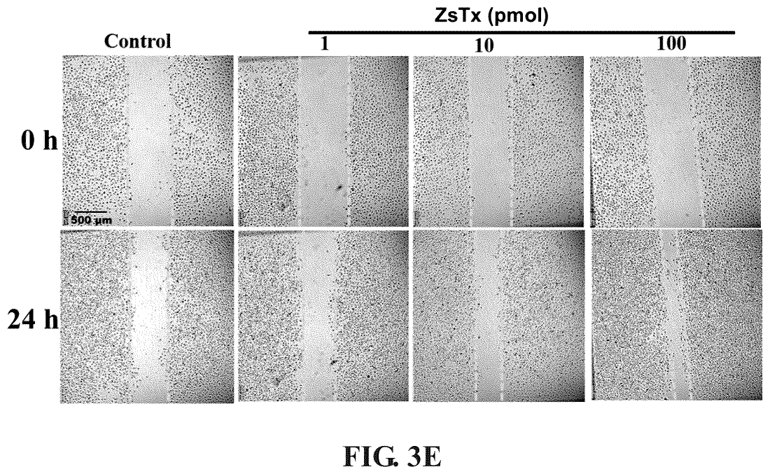

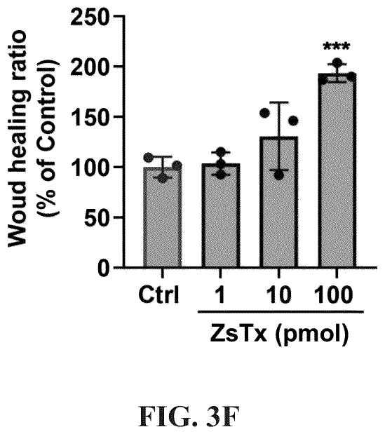

1. A toxicity test of the polypeptide ZsTx for HUVECs was conducted. The specific method was as follows: HUVECs were inoculated at a density of 5,000 cells/well in a 96-well plate, and cultivated at 5% CO₂ and 37° C. for 24 h. When a cell density reached 70%, cells were starved for 12 h in a low-serum medium. Then, the old medium was discarded, and the cells were co-incubated with ZsTx at different concentrations (0 μM to 1 μM) in a low-serum medium for 24 h. A cell viability was detected using a CCK8 kit. As shown in FIG. 3A, ZsTx has no toxicity for HUVECs at a concentration of 1 μM or less.
2. An impact of the polypeptide ZsTx on the proliferation of HUVECs was tested. The specific experimental method was as follows: HUVECs were inoculated at a density of 5,000 cells/well in a 96-well plate, and cultivated at 5% CO₂ and 37° C. for 24 h. When a cell density reached 70%, cells were starved for 12 h in a low-serum medium. Then, the old medium was discarded, and the cells were co-incubated with ZsTx at different concentrations (0 pmol to 100 pmol) in a complete serum medium for 24 h. A cell viability was detected using a CCK8 kit. As shown in FIG. 3B, ZsTx in a concentration range of 1 pmol to 100 pmol can promote the proliferation of HUVECs in a concentration-dependent manner.
3. An impact of the polypeptide ZsTx on the tube formation of HUVECs was tested. The specific experimental method was as follows: Matrigel pre-cooled at 4° C. was transferred at 100 μL/well to a 96-well plate and incubated in a 37° C. incubator for 30 min to establish a thin-layer barrier. Then, media including ZsTx at different concentrations (0 pmol to 100 pmol) were added at 50 μL/well to the Matrigel. An HUVEC suspension was added at 50 μL/well and 4×10⁴ cells/well to the Matrigel, and incubated at 37° C. for 4 h. A tube structure was photographed by an inverted microscope (LEICA BIOSYSTEMS) at a magnification of ×5×. A total tube length and a total branch length were analyzed with the Image J software. As shown in FIG. 3C and FIG. 3D, ZsTx in a concentration range of 1 pmol to 100 pmol can promote the tube formation of HUVECs in a concentration-dependent manner.
4. An impact of the polypeptide ZsTx on the migration of HUVECs was tested. The specific experimental method was as follows: HUVECs were inoculated at a density of 2×10⁵ cells/well evenly in a 24-well plate, and cultivated at 5% CO₂ and 37° C. for 24 h until a cell density reached 90%. The cell migration was determined by scratching a horizontal wound at a center of each well using a 200 μL pipette tip. The non-adherent cells were washed off with PBS, and then fresh media including ZsTx at different concentrations (0 pmol to 100 pmol) were added. Images for each well at 0 h and 24 h were recorded under an inverted microscope. A cell migration rate was analyzed with the ImageJ software. As shown in FIG. 3E and FIG. 3F, ZsTx in a concentration range of 1 pmol to 100 pmol can promote the migration of HUVECs in a concentration-dependent manner.

### Example 4 Verification of a Pro-Angiogenic Activity of the Polypeptide ZsTx in a Zebrafish Model In Vivo

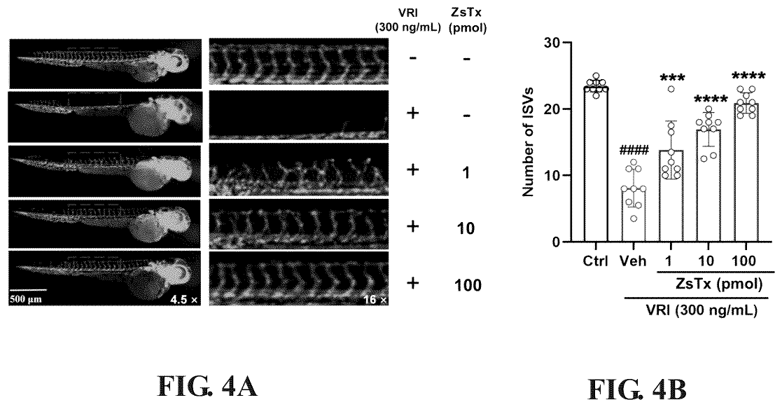

In this example, a zebrafish intersegmental vascular injury model was constructed to analyze a promotion effect of the active polypeptide ZsTx on the repair of damaged ISVs in zebrafish. The specific experimental method was as follows: 1-day-old zebrafish embryos of the transgenic Tg (fli-1: EGFP) line were taken, decoated, and randomly divided into 5 groups, namely, a control group, a model group, a low-concentration drug group, a medium-concentration drug group, and a high-concentration drug group. An E3 medium including VRI at 300 ng/mL was added to zebrafish embryos in the model group, the low-concentration drug group, the medium-concentration drug group, and the high-concentration drug group, and modeling was conducted for 3 h to construct a zebrafish vascular injury model. Then, the excess VRI was washed off. Zebrafish embryos successfully modeled were cultivated in an E3 medium, and the polypeptide ZsTx was added at 1 pmol, 10 pmol, and 100 pmol, respectively. On the second day, the growth of ISVs in zebrafish was observed under a fluorescence microscope, and a promotion effect of the polypeptide on the generation of blood vessels in zebrafish was analyzed. ISV index=intact blood vessels*1+mutilated blood vessels*0.5. As shown in FIG. 4A and FIG. 4B, VRI can significantly induce an intersegmental vascular injury in zebrafish, and ZsTx can repair an intersegmental vascular injury caused by VRI in a concentration-dependent manner.

### Example 5 Verification of Promotion of the Polypeptide ZsTx on the Healing of a Skin Wound in a Mouse Model In Vivo

In this example, a mouse wound model was constructed to analyze a promotion effect of the active polypeptide ZsTx on the healing of a skin wound in damaged mice. The specific experimental method was as follows: 12-week-old male BALB/c mice (with a body weight of 26 g to 30 g) were taken and intraperitoneally (i.p.) injected with sodium pentobarbital for surgical anesthesia. The mice each were shaved, and a skin wound (with a diameter of 8 mm) was created through full-thickness excision on a backside. The mice were randomly divided into 4 groups. The polypeptide ZsTx at specified concentrations (treatment groups) and PBS at an equal amount (control group) were applied evenly to a skin wound every day. In the study on wound healing, a change in a wound area was recorded and photographed on day 0, day 2, day 4, day 6, and day 8. The wound healing ratio was calculated as follows: wound healing ratio (%)=(A₀−A_{T})/A₀×100, where A₀ represents an initial wound area and Aₜ represents a wound area on day t after surgery. On day 8 after surgery, the mice were sacrificed, and skin samples were collected. The skin samples each were fixed with 10% formalin, treated, and embedded in paraffin. Skin sections were stained with Masson to investigate the collagen deposition reflecting a wound closure characteristic.

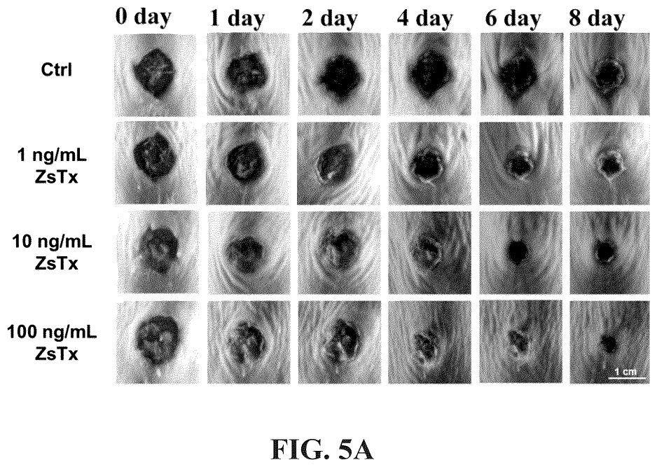

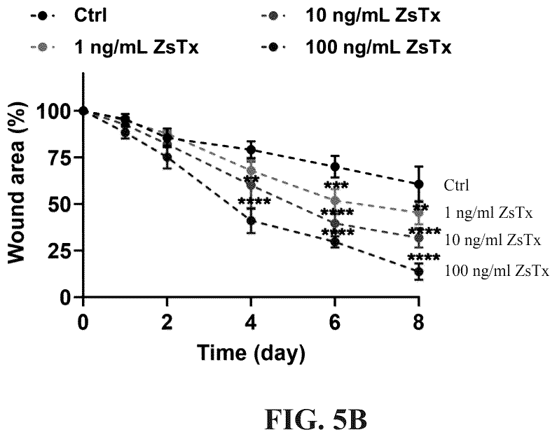

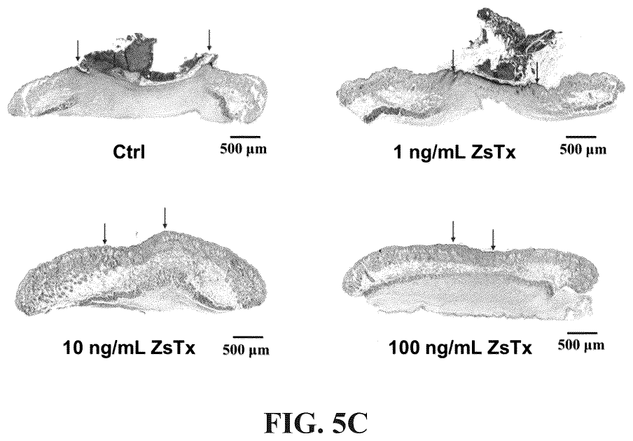

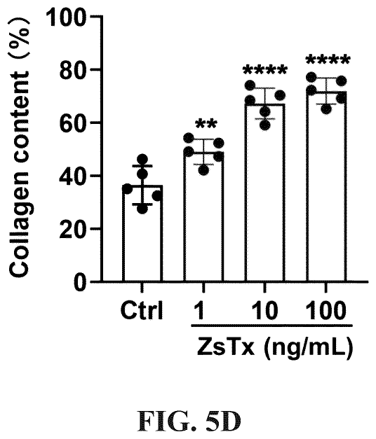

As shown in FIG. 5A and FIG. 5B, ZsTx can promote a wound healing process in mice, and wound areas measured on day 1, day 2, day 4, day 6, and day 8 after an injury are gradually reduced (FIG. 5A and FIG. 5B). In particular, wounds of mice treated with 100 ng/ml of ZsTx are almost closed on day 8, while a significant scar zone can still be detected in the control group. Masson staining results show that, compared with the control group, an increase in a number of collagen fibers is observed in a wound treated with ZsTx (FIG. 5C and FIG. 5D). The results show that the ZsTx treatment can accelerate a wound healing process in mice.

Finally, it should be noted that the above examples are provided merely to describe the technical solutions of the present application, rather than to limit the protection scope of the present application. Although the present application is described in detail with reference to preferred examples, a person of ordinary skill in the art should understand that modifications or equivalent replacements may be made to the technical solutions of the present application without departing from the spirit and scope of the technical solutions of the present application.

1. A polypeptide for promoting angiogenesis, comprising at least one selected from the group consisting of the following (a) to (c):
(a) a polypeptide in which a carboxyl terminus has a GRPRF (SEQ ID NO: 12) sequence;
(b) a polypeptide in which there is a GKR cleavage site at a carboxyl terminus; and
(c) a polypeptide in which there is an amidation modification at a carboxyl terminus.

2. The polypeptide for promoting angiogenesis according to claim 1, wherein the polypeptide is named ZsTx, and has an amino acid sequence set forth in SEQ ID NO: 1.

3. A nucleotide sequence encoding the polypeptide according to claim 2, wherein the nucleotide sequence encoding the polypeptide ZsTx is set forth in SEQ ID NO: 5.

4. A reagent for promoting angiogenesis, comprising the polypeptide ZsTx according to claim 2.

5. The reagent according to claim 4, wherein the reagent comprises a reagent that promotes proliferation, tube formation, and/or migration of human umbilical vein endothelial cells.

6. The reagent according to claim 4, wherein a use concentration of the polypeptide ZsTx is 1 pmol to 100 pmol.

7. A drug for preventing or treating a disease related to insufficient angiogenesis, comprising the polypeptide ZsTx according to claim 2.

8. The drug according to claim 7, wherein the disease related to insufficient angiogenesis comprises a cardiovascular disease, a peripheral artery disease, a wound healing disorder, tissue ischemia, and a coronary artery disease.

9. The drug according to claim 7, wherein a use concentration of the polypeptide ZsTx is 1 pmol to 100 pmol.

10. A pharmaceutical composition for promoting angiogenesis, comprising the polypeptide ZsTx according to claim 2 and a pharmaceutically acceptable carrier.

11. The pharmaceutical composition according to claim 10, wherein a use concentration of the polypeptide ZsTx is 1 pmol to 100 pmol.

12. A method for promoting angiogenesis, comprising: administering the polypeptide ZsTx according to claim 2 to endothelial cells cultivated in vitro; or administering the polypeptide ZsTx according to claim 2 to zebrafish or mice.

13. The method according to claim 12, wherein a concentration of the polypeptide ZsTx is 1 pmol to 100 pmol.

14. The method according to claim 12, wherein the zebrafish undergoes an intersegmental vascular injury.

15. The method according to claim 12, wherein the mice have a skin wound.


SEQUENCE LISTING (34 sequences)

SEQ ID NO: 1 - protein, 101 aa, Zoanthus sociatus
MRAPTLAFFWLVGVVIFSTFSKSHSQQHRIEAIQLMASLDPEILATLSGSAEYPDRPDVFKTPLQLRKYLESLNSFYALTGRPRFGKRRKRTTNDKPNEGQ

SEQ ID NO: 2 - protein, 73 aa, Zoanthus sociatus
MKTLFVFLIVAVFVANAAMYPPDRPDVFKTRADLRQYLEELNEFYAITCGRPRFGKRRKLSCLTQCDERGCLK

SEQ ID NO: 3 - protein, 96 aa, Zoanthus sociatus
MKLTCVLIIAVLFLTACQLTTAETYSRGRQKHRARRSTDKNSKWTRTYPDPPARPAVFHSREELMNYLRELNRYFAIVGRPRFGKRAILEIHVKSG

SEQ ID NO: 4 - protein, 93 aa, Zoanthus sociatus
MASVRILALLMIGTVLMCQVTANADSELLHEILSAHLEDNMPVKRMLAPPQRPEEFKSPNELRKYLKAINEYYAIVGRPRFGKRPALRLQARR

SEQ ID NO: 5 - DNA, 306 bp, Zoanthus sociatus
  source 1..306: mol_type = genomic DNA
atgcgagcacccactttagcctttttttggctagtgggggtggtgattttttcgactttttcaaaatctcattctcaacaacatcgaattgaagccattcaattgatggcttccttggaccccgaaattttggccacactttcaggttctgccgaatatccggacaggcctgatgttttcaaaacgcctctgcaattacggaaatatttggaatctctcaactcgttttacgctttgaccggtcgccctcgattcggaaaaagaagaaaacgaaccacaaatgacaaaccaaacgaaggccaatga

SEQ ID NO: 6 - DNA, 288 bp, Zoanthus sociatus
  source 1..288: mol_type = genomic DNA
atgaaactgacctgcgtgctgattattgcggtgctgtttctgaccgcgtgccagctgaccaccgcggaaacctatagccgcggccgccagaaacatcgcgcgcgccgcagcaccgataaaaacagcaaatggacccgcacctatccggatccgccggcgcgcccggcggtgtttcatagccgcgaagaactgatgaactatctgcgcgaactgaaccgctattttgcgattgtgggccgcccgcgctttggcaaacgcgcgattctggaaattcatgtgaaaagcggc

SEQ ID NO: 7 - DNA, 228 bp, Zoanthus sociatus
  source 1..228: mol_type = genomic DNA
atggcggaaacctatagccgcggccgccagaaacatcgcgcgcgccgcagcaccgataaaaacagcaaatggacccgcacctatccggatccgccggcgcgcccggcggtgtttcatagccgcgaagaactgatgaactatctgcgcgaactgaaccgctattttgcgattgtgggccgcccgcgctttggcaaacgcgcgattctggaaattcatgtgaaaagcggc

SEQ ID NO: 8 - DNA, 279 bp, Zoanthus sociatus
  source 1..279: mol_type = genomic DNA
atggcgagcgtgcgcattctggcgctgctgatgattggcaccgtgctgatgtgccaggtgaccgcgaacgcggatagcgaactgctgcatgaaattctgagcgcgcatctggaagataacatgccggtgaaacgcatgctggcgccgccgcagcgcccggaagaatttaaaagcccgaacgaactgcgcaaatatctgaaagcgattaacgaatattatgcgattgtgggccgcccgcgctttggcaaacgcccggcgctgcgcctgcaggcgcgccgc

SEQ ID NO: 9 - protein, 25 aa, Zoanthus sociatus
MRAPTLAFFWLVGVVIFSTFSKSHS

SEQ ID NO: 10 - protein, 28 aa, Zoanthus sociatus
QQHRIEAIQLMASLDPEILATLSGSAEY

SEQ ID NO: 11 - protein, 32 aa, Zoanthus sociatus
PDRPDVFKTPLQLRKYLESLNSFYALTGRPRF

SEQ ID NO: 12 - protein, 5 aa, Zoanthus sociatus
GRPRF

SEQ ID NO: 13 - protein, 35 aa, Armadillidium vulgare
AQYPDRPDVFKTPADLRQYLEELNSFYAITGRPRF

SEQ ID NO: 14 - protein, 34 aa, Haliotis discus
APPDRPSEFRSPDQLRQYLKALNEYYAIVGRPRF

SEQ ID NO: 15 - protein, 39 aa, Lymnaea stagnalis
TEAMLTPPQRPEEFKNPNELRKYLKAINEYYAIVGRPRF

SEQ ID NO: 16 - protein, 36 aa, Idiosepius paradoxus
MFAPPNRPAEFKSPEELRQYMKALNEYYAIVGRPRF

SEQ ID NO: 17 - protein, 39 aa, synthetic construct
  VARIANT 1..2: note = X is A or R or N or D or C or Q or E or G or H or I or L or K or M or F or P or S or T or W or Y or V
  VARIANT 4..7: note = X is A or R or N or D or C or Q or E or G or H or I or L or K or M or F or P or S or T or W or Y or V
  VARIANT 12: note = X is A or R or N or D or C or Q or E or G or H or I or L or K or M or F or P or S or T or W or Y or V
  VARIANT 15..16: note = X is A or R or N or D or C or Q or E or G or H or I or L or K or M or F or P or S or T or W or Y or V
  VARIANT 18: note = X is A or R or N or D or C or Q or E or G or H or I or L or K or M or F or P or S or T or W or Y or V
  VARIANT 25: note = X is A or R or N or D or C or Q or E or G or H or I or L or K or M or F or P or S or T or W or Y or V
  VARIANT 34: note = X is A or R or N or D or C or Q or E or G or H or I or L or K or M or F or P or S or T or W or Y or V
XXAXXXXPERPXEFXXPXELRKYLXALNEYIAIXGRPRF

SEQ ID NO: 18 - protein, 40 aa, Aplysia californica
DNSEMLAPPPRPEEFTSAQQLRQYLAALNEYYSIMGRPRF

SEQ ID NO: 19 - protein, 39 aa, Helix aspersa
STQMLSPPERPREFRHPNELRQYLKEINEYYAIMGRTRF

SEQ ID NO: 20 - protein, 37 aa, Conus sp.
TVSDPPARPAVFHSREELMNYVRELNRYFAIVGRPRF

SEQ ID NO: 21 - protein, 37 aa, Conus sp.
TVSDPPARPAVFHSREELMNYVRELNLYFAIVGRPRY

SEQ ID NO: 22 - protein, 39 aa, Moniezia sp.
PDKDFIVNPSDLVLDNKAALRDYLRQINEYFAIIGRPRF

SEQ ID NO: 23 - protein, 39 aa, Moniezia sp.
PDQDAIVNPSDLVLDNKAALRDYLRQINEYFAIIGRPRF

SEQ ID NO: 24 - protein, 36 aa, Danio rerio
YPTKPDNPGEDAPAEELAKYYSALRHYINLITRQRY

SEQ ID NO: 25 - protein, 36 aa, Torpedo marmorata
YPSKPDNPGEGAPAEDLAKYYSALRHYINLITRQRY

SEQ ID NO: 26 - protein, 36 aa, Squalus acanthias
YPPKPENPGEDAPPEELAKYYSALRHYINLITRQRY

SEQ ID NO: 27 - protein, 36 aa, Pelophylax ridibundus
YPSKPDNPGEDAPAEDMAKYYSALRHYINLITRQRY

SEQ ID NO: 28 - protein, 36 aa, Bos taurus
YPSKPDNPGEDAPAEDLARYYSALRHYINLITRQRY

SEQ ID NO: 29 - protein, 36 aa, Rattus norvegicus
YPSKPDNPGEDAPAEDMARYYSALRHYINLITRQRY

SEQ ID NO: 30 - protein, 36 aa, Oryctolagus cuniculus; identical to SEQ ID NO: 29

SEQ ID NO: 31 - protein, 36 aa, Cavia porcellus; identical to SEQ ID NO: 29

SEQ ID NO: 32 - protein, 36 aa, Gallus gallus
YPSKPDSPGEDAPAEDMARYYSALRHYINLITRQRY

SEQ ID NO: 33 - protein, 36 aa, Homo sapiens; identical to SEQ ID NO: 29

SEQ ID NO: 34 - protein, 8 aa, synthetic construct
  VARIANT 3: note = X is A or R or N or D or C or Q or E or G or H or I or L or K or M or F or P or S or T or W or Y or V
  VARIANT 5: note = X is A or R or N or D or C or Q or E or G or H or I or L or K or M or F or P or S or T or W or Y or V
GRXRXGKR